(19) United States
(12) Patent Application Publication
(10) Pub. No.: US 2025/0216393 A1
(43) Pub. Date: Jul. 3, 2025

(54) PLASMA AUTOANTIBODY BIOMARKERS FOR DIAGNOSIS OF LUNG CANCER

(71) Applicants: ARIZONA BOARD OF REGENTS ON BEHALF OF ARIZONA STATE UNIVERSITY, Scottsdale, AZ (US); NEW YORK UNIVERSITY, New York, NY (US)

(72) Inventors: Joshua Labaer, Chandler, AZ (US); Ji Qiu, Chandler, AZ (US); Jie Wang, Tempe, AZ (US); Garrick Wallstrom, Mesa, AZ (US); Jin Park, Phoenix, AZ (US); William Rom, New York, NY (US); Harvey Pass, New York, NY (US)

(21) Appl. No.: 18/942,134

(22) Filed: Nov. 8, 2024

Related U.S. Application Data
(63) Continuation of application No. 16/069,111, filed on Jul. 10, 2018, now abandoned.
(60) Provisional application No. 62/277,786, filed on Jan. 12, 2016.

(51) Int. Cl. G01N 33/574; G01N 33/564

(52) U.S. Cl. CPC G01N 33/57484; G01N 33/564; G01N 33/57423; G01N 2800/56; G01N 2800/60

(57) ABSTRACT

An immune-proteomic screening of AAb responses using protein arrays has identified two panels of Aab (antigen/antibody complexes) that can potentially differentiate lung adenocarcinoma from smoker controls as well as CT positive benign lung disease. The resulting biomarkers appear to have high specificity so that high risk subjects with a positive CT screen and a positive serum test should get more invasive test such as needle biopsy for a timely cancer diagnosis, among other advantages.

### CROSS-REFERENCE TO RELATED APPLICATIONS

This application is a continuation of U.S. application Ser. No. 16/069,111, filed Jul. 10, 2018, which represents the U.S. National Stage entry of PCT/US2017/013037, filed Jan. 11, 2017, and claims the benefit of U.S. Provisional Patent Application No. 62/277,786 filed Jan. 12, 2016, each of which are incorporated by reference herein in their entireties.

### STATEMENT REGARDING FEDERALLY SPONSORED RESEARCH

This invention was made with government support under U01 CA117374 and U01 CA086137 awarded by the National Institutes of Health. The government has certain rights in the invention.

### FIELD OF THE INVENTION

This disclosure relates to biomarker complexes and detection in the field of lung cancer.

### BACKGROUND OF THE INVENTION

The reduction in lung cancer mortality associated with CT screening has led to its increased use and a concomitant increase in the detection of benign pulmonary nodules. Many of these individuals undergo unnecessary, costly and invasive procedures. Therefore, there is a need for companion diagnostics that stratify individuals with pulmonary nodules into high risk or low risk groups.

Lung cancer has long been the leading cause of cancer deaths in the United States, with more than 150,000 deaths in year 2014. 5 year survival rate of lung cancer overall is only 17%, and 57% of lung cancers are diagnosed at advanced stage with 5 year survival rate as low as only 4%. Currently, low-dose computed tomography (CT) scans are used to screen populations with extensive smoking history between 55 and 74 years old. CT scan has been proven to effectively reduce lung cancer mortality by 20%, but many of the millions of pulmonary nodules identified by CT remained undiagnosed as malignant or benign.

According to the National Lung Screening Trial (NLST), only 3.6% of the nodules detected by CT were confirmed to have lung cancer, suggesting a high false positive rate. Therefore, there is a need for diagnostic tests that differentiate malignant from benign nodules, improving the diagnostic performance when combined with CT screening.

### SUMMARY OF THE INVENTION

We have performed one of the first studies in understanding humoral immune response in cancer patients, patients with benign nodules, and healthy smokers. We first profiled sero-reactivity to 10,000 full-length human proteins in 40 patients with early stage lung cancer and 40 smoker controls using nucleic acid programmable protein arrays (NAPPA) to identify candidate cancer-specific autoantibodies (AAbs). ELISA assays of promising candidates were performed on 137 lung cancer patients and 127 smoker controls as well as 170 subjects with benign pulmonary nodules. From protein microarray screening experiments, using a discovery set of 40 patients and 40 smoker controls, seventeen antigens showing higher reactivity in lung cancer cases relative to controls were subsequently selected for evaluation in a large sample set (n=264) using enzyme-linked immunosorbance assay (ELISA).

A previously unknown 5-AAb classifier (TTC14, BRAF, ACTL6B, MORC2, CTAG1B) was developed that can differentiate lung cancers from smoker controls with a sensitivity of 30% at 89% specificity. We further tested AAb responses in subjects with CT positive benign nodules (n=170), and developed a 5-AAb panel (KRT8, TTC14, KLF8, BRAF, TLK1) with a sensitivity of 30% at 88% specificity. Interestingly, mRNA levels of 6 AAb targets (TTC14, BRAF, MORC2, CTAG1B, KRT8, TLK1) were also found to increase in lung adenocarcinoma tissues based on the TCGA data set. Thus, we discovered previously unknown antibody/antigen complexes, i.e., Aabs, associated with lung adenocaricnoma, which have potential to differentiate cancer from CT positive benign diseases.

### BRIEF DESCRIPTION OF THE DRAWINGS

FIG. 1. Study Design.

FIGS. 2A-2B. Summary of lung cancer associated antigens discovered from protein array screening, with (A) showing a GO enrichment analysis of lung cancer associated AAb targets selected from protein array screening. Term embryonic morphogenesis has 12 genes, whereas the rest of the node has 3 to 4 genes each. (B) shows a heatmap of differential AAb responses in lung cancer compared to smoker controls.

FIG. 3. Responses of individual AAb from panel II. Individual cutoffs at 98 percentile of benign controls were drawn as solid lines.

FIGS. 4A-4B. Multivariate analysis of clinical factors and AAb responses. A. Analysis of smoking history, AAb responses and nodule size in lung cancer and benign control. B. Analysis of smoking history, stage and AAb responses in lung cancer cases. (A and B, Smoking is measured by pack-year on the vertical axis. Nodule size is presented by circle diameter.)

FIG. 5. mRNA expression level of AAb targets from both panels (TCGA). Only mRNA levels in lung adenocarcinoma (ADC) and solid tissue normal (Normal) were graphed.

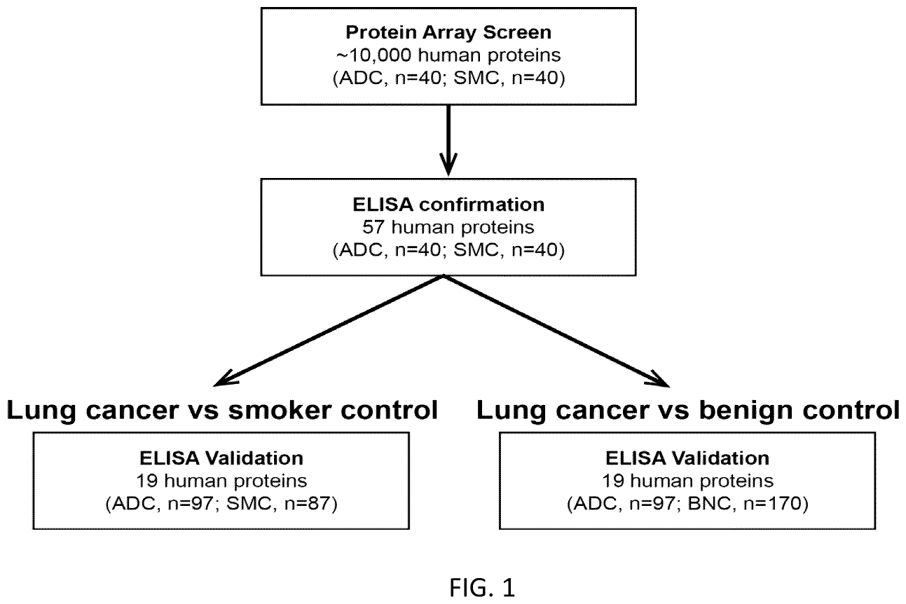

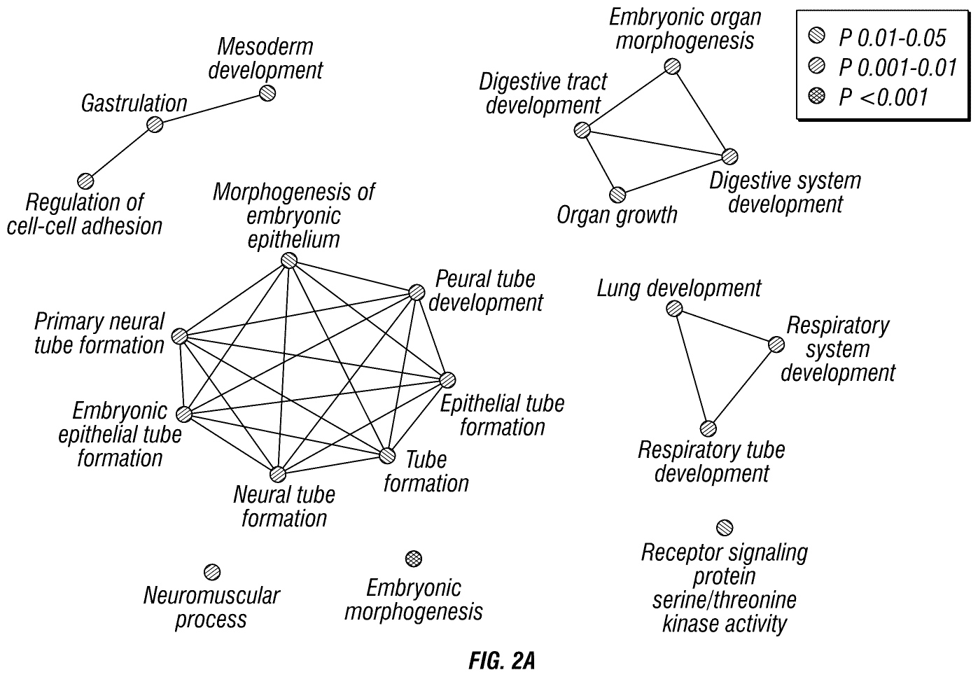

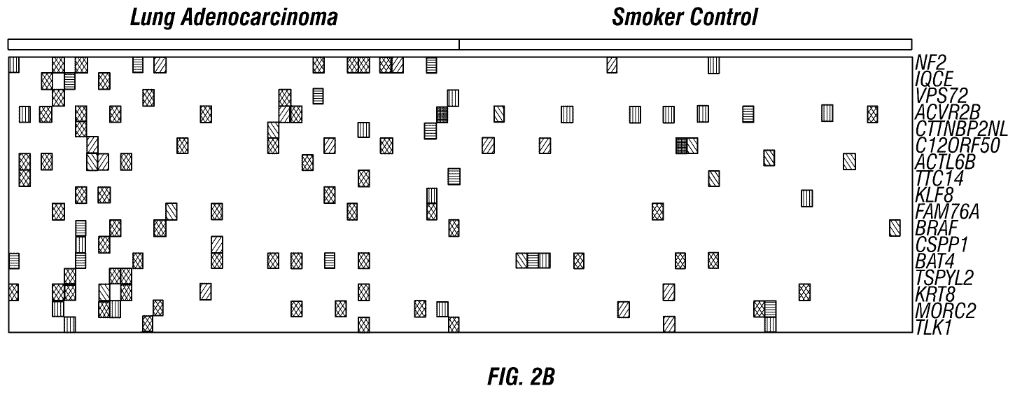

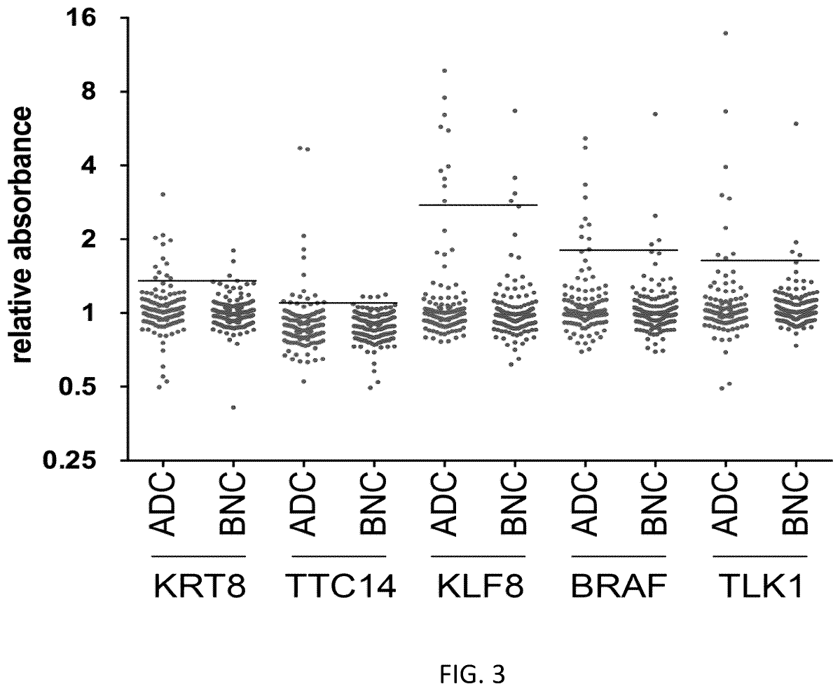

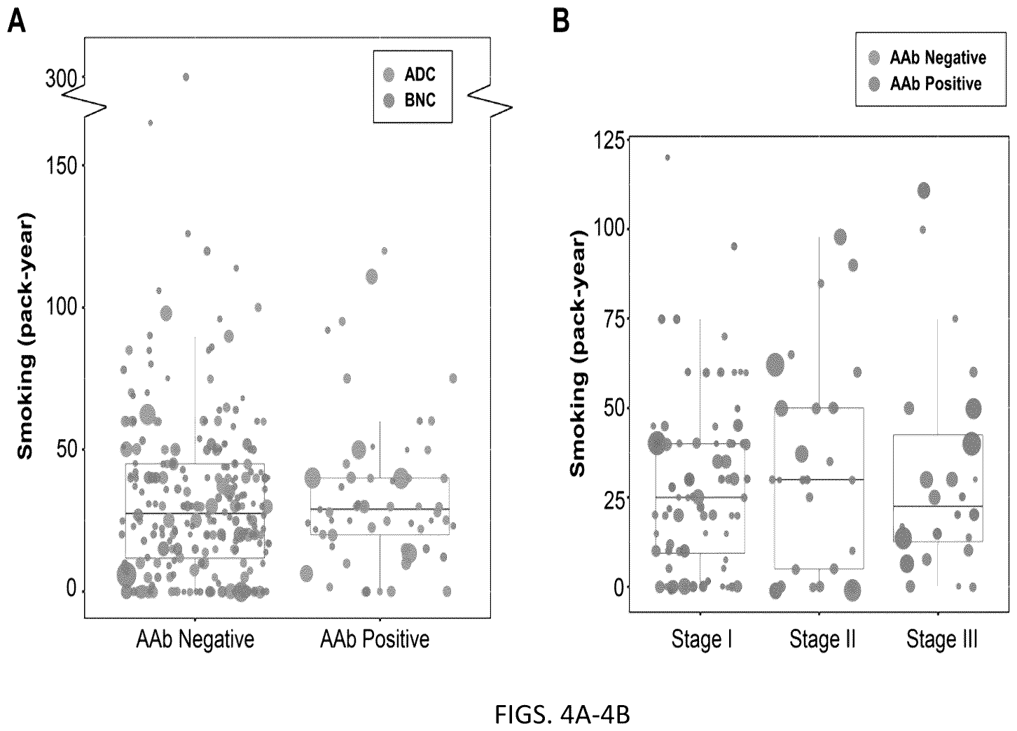

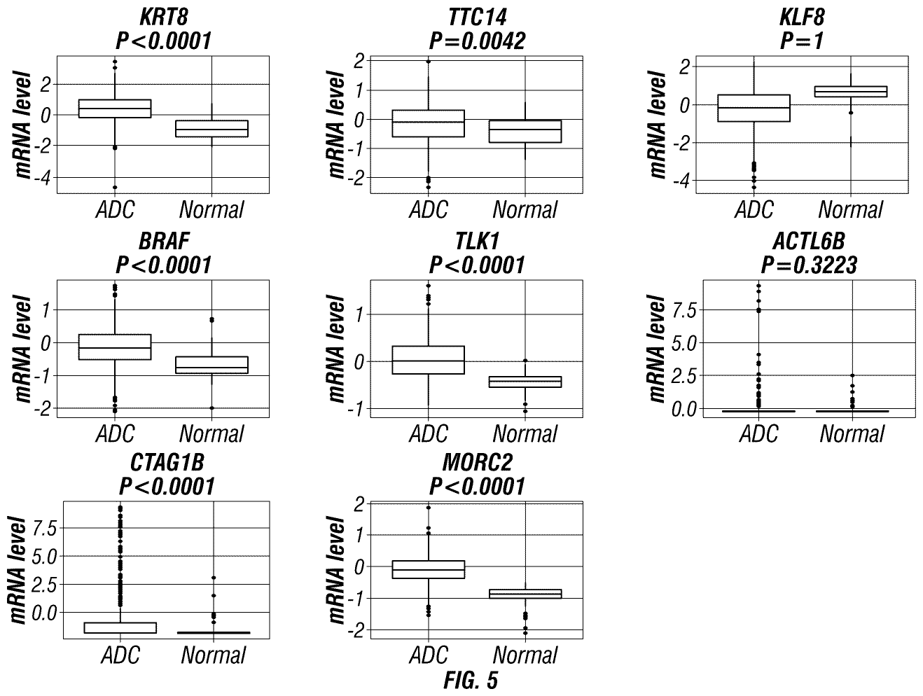

### DETAILED DESCRIPTION OF THE INVENTION

Lung cancers can trigger host immune responses and elicit antibodies against tumor antigens. The identification of the disclosed autoantibodies (AAbs) and their corresponding antigens impact our knowledge of cancer immunity, leading to early diagnostics or even benefiting immunotherapy.

Previous studies were mostly performed in the context of comparing cancers and healthy (smoker) controls. Practically, tests for such markers should rely on readily accessible samples, like plasma or sputum, because they are likely to be performed on individuals undergoing screening. Tremendous efforts have been spent on the identification of proteins, circulating tumor cells, circulating tumor DNAs, and circulating miRNA for this purpose.

The concentration of many molecular markers in blood tends to be very low because it relies upon secretion by cancer cells, which are few in number in the pre-clinical stage. Typically, only a fraction of the secreted biomarker gets distributed to the plasma where the biomarker gets diluted in a large volume in blood. These low concentrations make discovery and routine detection challenging.

An alternative strategy is to exploit the ability of the immune system to detect the presence of tumor cells through the generation of autoantibodies (AAb). These responses of the adaptive immune system against target tumor antigens effectively amplify the signals from the minute amount of tumor proteins released from cancer tissue.

Here, we describe one of first studies at proteome scale, which focuses on comparing plasma AAb responses in a homogenous sample set of lung adenocarcinoma (ADC) patients, heavy smoker subjects (SMC), as well as benign nodules controls (BNC). We focused on a single subtype of lung cancer to avoid heterogeneity and enable us to exclude between-subtype variation. We started with an unbiased screen for cancer-specific antibodies in patients with the adenocarcinoma subtype non-small cell lung cancer (NSCLC) and age, gender, smoking matched controls, using nucleic acid programmable protein arrays (NAPPA) displaying ˜10,000 full length human proteins. Candidate lung cancer-specific antibodies were further assessed in an independent set of cases and controls, including subjects with benign pulmonary nodules.

The 19 antigens that we have identified as potential biomarkers for the early detection of lung cancer are: TTC14, VPS72, CTTNBP2NL, TSPYL2, ACTL6B, ACVR2B, BRAF, KLF8, BAT4, C12ORF50, IQCE, CSPP1, KRT8, MORC2, FAM76A, NF2, TLK1, P53 (TP53), NYESO1 (CTAG1B). We identified an AAb panel to differentiate lung cancer patients from matched smoker controls with 30% sensitivity at 89% specificity. In addition, we also identified an AAb panel to differentiate lung cancer patients from matched low-dose computed tomography positive benign controls with 30% sensitivity at 88% specificity. We designated samples as positive if they exceeded antigen specific cutoffs for any 1 of the AAb from the panel. Aab specific cutoffs were set at the level of 98% specificity for each AAb.

TABLE 1
Discovery and validation statistics of selected AAbs.

| | Discovery | | Validation | | Validation | |
|---|---|---|---|---|---|---|
| | (ADC, n = 40; | | (ADC, n = 97; | | (ADC, n = 97; | |
| | SMC, n = 40) | | SMC, n = 87) | | BNC, n = 170) | |
| Antigen | Sensitivity | Specificity | Sensitivity | Specificity | Sensitivity | Specificity |
| TTC14 | 17.5% | 97.5% | 11.3% | 97.7% | 5.2% | 97.6% |
| VPS72 | 17.5% | 97.5% | 0.0% | 97.7% | 2.1% | 97.6% |
| CTTNBP2NL | 15.0% | 97.5% | 3.1% | 97.7% | 2.1% | 97.6% |
| TSPYL2 | 15.0% | 97.5% | 2.1% | 97.7% | 1.0% | 97.6% |
| ACTL6B | 15.0% | 97.5% | 3.1% | 97.7% | 2.1% | 97.6% |
| ACVR2B | 15.0% | 97.5% | 4.1% | 97.7% | 2.1% | 97.6% |
| BRAF | 12.5% | 97.5% | 5.2% | 97.7% | 6.2% | 97.6% |
| KLF8 | 12.5% | 97.5% | 1.0% | 97.7% | 5.2% | 97.6% |
| BAT4 | 12.5% | 97.5% | 0.0% | 97.7% | 0.0% | 97.6% |
| C12ORF50 | 10.0% | 97.5% | 2.1% | 97.7% | 2.1% | 97.6% |
| IQCE | 10.0% | 97.5% | 4.1% | 97.7% | 4.1% | 97.6% |
| CSPP1 | 7.5% | 97.5% | 1.0% | 97.7% | 0.0% | 97.6% |
| KRT8 | 7.5% | 97.5% | 0.0% | 97.7% | 7.2% | 97.6% |
| MORC2 | 7.5% | 97.5% | 4.1% | 97.7% | 1.0% | 97.6% |
| FAM76A | 7.5% | 97.5% | 1.0% | 97.7% | 2.1% | 97.6% |
| NF2 | 5.0% | 97.5% | 2.1% | 97.7% | 2.1% | 97.6% |
| TLK1 | 5.0% | 97.5% | 4.1% | 97.7% | 6.2% | 97.6% |
| TP53 | 2.5% | 97.5% | 3.1% | 97.7% | 4.1% | 97.6% |
| CTAG1B | 2.5% | 97.5% | 9.3% | 97.7% | 3.1% | 97.6% |

TABLE 2
Sensitivity and specificity of individual AAb from panel I.

Overall (ADC, n = 137; SMC, n = 127)

| Antigen | Sensitivity | Specificity |
|---|---|---|
| TTC14 | 12.4% | 97.6% |
| BRAF | 8.0% | 97.6% |
| ACTL6B | 5.1% | 97.6% |
| CTAG1B | 5.1% | 97.6% |
| MORC2 | 5.1% | 97.6% |

TABLE 3
Sensitivity and specificity of individual AAb from panel II.

Overall (ADC, n = 137; BNC, n = 170)

| Antigen | Sensitivity | Specificity |
|---|---|---|
| KRT8 | 8.8% | 97.6% |
| TTC14 | 8.0% | 97.6% |
| KLF8 | 7.3% | 97.6% |
| BRAF | 6.6% | 97.6% |
| TLK1 | 5.8% | 97.6% |

Using an immuno-proteomics approach, we profiled antibody responses in healthy, heavy-smoker controls and lung cancer patients. Bioinformatics analysis revealed significantly enriched pathways related to embryonic morphogenesis, organ development (including lung development) and receptor signaling and serine/threonine kinase pathways. The performance of a subset of antibodies was confirmed by ELISA using an expanded sample set including subjects with benign nodules, with sensitivities ranging from 5-10% at 98% specificity. We reported a 5-AAb panel (TTC14, BRAF, ACTL6B, MORC2, CTAG1B) that had 30% sensitivity at 89% specificity to distinguish lung cancer from high-risk controls with smoking histories.

A comparison of AAb responses between lung cancer and patients with CT positive pulmonary nodules revealed a related but different 5-AAb panel (TTC14, BRAF, KLF8, TLK1, KRT8) with a sensitivity of 30% at 88% specificity. We also calculated the sensitivities and specificities of all 19 candidate antigens that went into the validation study restricting benign samples with above or equal to 30 pack-year smoking history. All five reported candidate antigens still presented sensitivities above 5% at 98% specificity showing that they still provide discrimination between lung adenocarcinoma and benign controls (not shown).

Although these panels require further validation, they do provide information on the complementarities of these informative antigens. Further analysis revealed that AAbs do not associate with tumor size, stage, or smoking history. To our knowledge, this is one of the first studies that applied an immuno-proteomics approach on the identification of specific antibodies that might help stratify subjects with positive CT nodules into benign lung disease controls and lung adenocarcinoma patients. To ensure accurate estimations of responses when analyzing ELISA results, we also estimated the background associated with the supporting reagent for each plasma sample, which provides the most rigorous assay in similar studies.

In view of the above, one point of novelty is the identification of the 19 antigens as biomarkers for early detection of lung cancer. Many of them have not been previously associated with lung cancer. In addition, we also developed panels to differentiate patients with lung cancer from either matched smoker controls with 30% sensitivity at 89% specificity; or matched CT screen positive benign controls with 30% sensitivity at 88% specificity.

Currently, there is no clinically available blood test that can differentiate lung cancer patients from computed tomography positive populations. Moreover, complexes of Aab's and the antigens described herein are believed to be novel in the context of lung cancer detections and patient differentiation.

### Non-Limiting Examples

### Characteristics of Plasma Samples

A total of 434 plasma samples were obtained from NYU with 137 lung adenocarcinoma, 127 controls with smoking history, and 170 benign pulmonary nodules (granuloma, n=47; emphysema, n=50; stable nodules, n=73). All lung cancer samples were collected in the operating room at the time of surgery and were pathologically confirmed. None had any prior treatment. EDTA plasma samples were processed within 4 hours of harvest and frozen at −80° C.

The patients with adenocarcinoma were recruited from the clinics at the NYU Cancer Center and all gave informed consent for the IRB approved Lung Cancer Biomarker Center Lung Cancer Protocol #8896. Our control samples contain high-risk smokers who were recruited into the NYU Lung Cancer Biomarker Center. The mean smoking history in this cohort was 42 pack-years. We also matched on smoking with the lung cancer subjects who contained some never smokers. All recruitment of control samples was IRB approved. This was a voluntary recruitment with letters, phone calls, visits to unions primary care physicians and Con Edison by Dr. Rom and his study nurse. Lung cancer patients were referred for evaluation of a nodule. Lung nodule patients were referred to Dr. Harvey Pass for evaluation. The NYU Lung Cancer Biomarker Center performs low dose CT-scan screening for high-risk smokers as part of the National Cancer Institute's Early Detection Research Program.

Benign nodules are followed for two years and had no growth. None of the study subjects had previous cancer or chemotherapy. All subjects had blood drawn by EDRN protocol, spirometry, and questionnaires with smoking history collected. Staging of lung cancer was according to the IASLC protocols.

In the discovery sample set for protein array experiment, 40 patients with lung adenocarcinoma were matched to 40 cancer free controls by age, gender and smoking history. 38 out of 40 patients from the discovery samples had stage I disease. For validation purpose, additional 97 patients with lung adenocarcinoma of different stages (47% stage I) and 87 controls as well as 170 patients with CT positive benign lung disease were included.

### Protein Array Experiments

Open reading frames were obtained from DNASU (https://dnasu.org/). Production of the protein array and array quality control experiments were performed as previously described. In brief, arrays displaying 10,000 human proteins (distributed evenly on five array sets) were manufactured. Plasma probing experiments were performed using HS 4800™ Pro hybridization station (Tecan). Briefly, slides were first incubated with SuperBlock (Pierce), and then proteins were expressed using 1-Step Human Coupled in vitro Expression system (Thermo). After blocking with 5% milk in phosphate buffered saline with Tween 20 (milk-PBST), slides were incubated with plasma samples (1:50, pre-incubated 2-3 h with 5% milk prepared with E. coli lysate) for 16 h at 4° C., followed by 3 times wash with 5% milk-PBST. Then slides were incubated with Dylight649 labeled goat anti-human IgG (Jackson ImmunoResearch Laboratories) at 23° C. for 1 h. Slides were then washed, dried and scanned by Tecan scanner under consistent settings.

### Protein Array Image Analysis and Quantification

The scanned protein array images were examined using ArrayPro Analyzer (MediaCybernetics). To capture real antibody responses that cannot be quantified by the image analysis software, two researchers qualitatively examined all images to identify and confirm positive responses, which were described previously. Briefly, raw images were adjusted to extreme contrast and brightness using ArrayPro Analyzer (MediaCybernetics), and each spot was graded at a scale of 0 to 5 based on ring's intensity and morphology.

### Candidate Selection

Protein antigens were selected for subsequent ELISA confirmation when they showed higher prevalence in lung adenocarcinoma based on visual analysis. Specifically, they had to meet all of the following criteria: 1). Their frequency in ADC minus frequency in SMC is greater than or equal to 2; 2). Frequency in ADC divided by frequency in SMC is greater than or equal to 1.4. Totally, 57 protein antigens were selected.

### Pathway Analysis

Gene Ontology term enrichment analysis was performed using Cytoscape with ClueGo plugins on all 57 proteins with customized reference of all proteins displayed on our protein array. Gene symbol was used as identifier for the analysis. Node size was set proportional to number of genes observed. Node color was coded to reflect Benjamini-Hochberg adjusted p value.

### ELISA Assays

ELISA assays were performed to verify selected AAb responses towards protein antigens using freshly produced human proteins as previously described. In brief, 96-well highbind ELISA plates (Corning) were coated with goat anti-GST antibody (GE Healthcare) at 10 μg/ml in 0.2 M sodium bicarbonate buffer pH9.4 overnight at 4° C. 1 day prior to experiment. All high-throughput liquid handling were performed using a BioMek NxP Laboratory Automation Workstation (Beckman Coulter).

Proteins were produced using hela lysate in vitro transcription-translation system (Thermo Scientific), and then captured on 5% milk-PBST blocked, GST antibody-coated ELISA plate. Plasma sample were then diluted at 1:200 in 5% milk-PBST, and incubated for 1 hr at room temperature with shaking. Horseradish peroxidase conjugated anti human IgG were used as secondary antibody (Jackson Laboratory). Plates were then developed by addition of TMB substrate (Thermo Scientific) for 15 minutes, and stopped by addition of 2M sulfuric acid. OD450 were measured using Perkin Elmer plate reader. ELISA relative absorbance of each plasma sample-antigen reaction (complex) was calculated using OD450 of expressed antigens over the median OD450 of all antigens measured for that sample. The median value was used to normalize systematic background of each plasma sample.

### Statistics and Data Analysis

To combine AAbs into panels, we used the 98 percentiles of the relative absorbance of either smoker control subjects (Panel I) or benign control subjects (Panel II) as cutoffs. A sample is called positive for lung adenocarcinoma if the AAb responses to one of the panel candidates exceed its corresponding cutoff.

A heatmap was developed to display differential AAb responses of 17 selected targets in lung cancer patients and smoker controls using the confirmation ELISA results. The heatmap color was scaled according to each AAb, and constructed using the gplots package in R.

We categorized subjects as AAb responders from ELISA analysis of each antigen if they exceed the 98-percentile of values from benign subjects. We constructed a multivariate logistic regression model to examine the association of AAb responses to age, nodule size and smoking history in lung cancer patients and benign controls. A second multivariate logistic regression added lung cancer status as an independent variable to further assess the relationship between nodule size, lung cancer status and AAb responses. We also constructed a multivariate logistic regression model to analyze the association of AAb responses to tumor size, node status, and tumor stage among lung cancer patients.

To compare TCGA mRNA expression levels between lung adenocarcinoma and normal tissues, we used one-sided Welch's t test. The TCGA lung adenocarcinoma data were generated by Illumina HiSeq, and obtained from UC Santa Cruz Cancer Genome Browser (https://genome-cancer.ucsc.edu/) TCGA_LUNG_exp_HiSeqV2-2014-08-22. All intensities were normalized by subtracting the mean value of each mRNA from each sample.

Identification of Candidate AAbs Associated with Lung Adenocarcinoma

To identify lung adenocarcinoma-associated candidate AAbs, we first performed comprehensive profiling of antibodies against 10,000 full-length human proteins in plasma samples from 40 patients with lung adenocarcinoma and 40 heavy smoker controls on NAPPA. Based on the array data, we selected 57 antigens whose AAb responses were differentially presented in lung cancer patients compared to smoker controls. A gene ontology enrichment analysis of these 57 candidate AAb targets revealed their involvement in embryonic morphogenesis, organ development, kinase signaling, and intermediate filament cytoskeleton. We then assessed these selected candidates by ELISA using the same samples. Based on ELISA, 17 antigens were confirmed to elicit differential AAb responses in lung cancer patients, and included for subsequent analysis.

### Validation in Lung Cancer Patients Versus Healthy Smoker Controls

To verify the levels of these 17 AAbs in lung cancer patients, we measured these AAbs in 184 additional plasma samples from 97 cases and 87 controls. In addition, we also included TP53 and CTAG1B proteins as possible candidates according to previous publications. Sero-positivity cutoffs of individual AAbs were set at 98 percentile of the ELISA absorbance in the 87 control samples. AAbs to TTC14, BRAF, and CTAG1B had sensitivity above 5% at 98% specificity when comparing lung cancer patients with smoker controls. In addition, Sensitivities of AAbs to TTC14, BRAF, ACTL6B, MORC2 and CTAG1B were above 5% at 98% specificity in the entire sample set. Further analysis of these 5 antigens using a standardized cutoff for each antigens of a relative absorbance greater than or equal to the 98 percentile of the relative absorbance in smoker controls, revealed a 5-AAb panel (panel I) with 30% sensitivity and 89% specificity.

### Classification of Lung Cancer Versus Benign Controls

To test whether these 17 AAb together with AAb against TP53 and CTAG1B can differentiate lung cancer from benign disease identified by CT screening, we analyzed the AAb responses against these antigens from 267 plasma samples by ELISA. As above, cutoffs of individual AAb were set at 98 percentile of the relative absorbance in benign controls. KRT8, TTC14, KLF8, BRAF, TLK1 were confirmed for their association to lung cancer patients compared with benign controls. They also had overall sensitivity above 5% at 98% specificity.

Further analysis of these 5 antigens using a standardized cutoff for each antigen of a relative absorbance greater than or equal to the 98 percentile of the relative absorbance in benign controls, revealed a 5-AAb panel (panel II) with 30% sensitivity and 88% specificity. We then evaluated these candidates AAbs' ability to distinguish lung cancer from benign controls with over 30 pack-year smoking history to mimic the intended population. In addition to the above five AAbs, NF2 and CTTNBP2NL AAbs were also presented sensitivity above 5% at 98% specificity. Sensitivities of individual AAb using subjects with different benign lung nodules were also assessed.

### Effect of Patient and Disease Characteristics on AAb Positivity

We compared clinical risk factors of smoking history, tumor size, gender and age to the AAb responses. Using the defined cutoffs derived from panel II, there were no significant differences in gender (P=0.212), age (P=0.818) or smoking history (P=0.635) between AAb responders and nonresponders, whereas nodule size was found to be significantly associated with AAb responses (P=0.025). As benign controls were not matched to cases according to nodule size, after adjusting for disease status, the association was no longer significant (P=0.752).

This result indicated that the AAb panels provide additional information on lung cancer status and that the observed AAb responses were independent of the known risk factors. In addition, we also analyzed the association between AAb responses among lung cancer patients and patients' characteristics including smoking history, tumor size, node status, and tumor stage. No significant association was observed between these patient characteristics and AAb responses. While not statistically significant, we observed that TTC14 AAb had higher prevalence in stage I lung cancer, whereas AAb against BRAF had higher prevalence in stage II and III.

Correlation of AAb Targets and their mRNA Level

We further investigated the tissue mRNA levels of protein antigens from both panels using TCGA data. 6 out of 8 proteins showed significantly increased expression in lung adenocarcinoma tissues compared to normal tissue. This orthogonal analysis confirmed our discovery of these AAbs' association with lung adenocarcinoma.

Using an immuno-proteomics approach, we profiled antibody responses in healthy, heavy-smoker controls and lung cancer patients. Bioinformatics analysis revealed significantly enriched pathways related to embryonic morphogenesis, organ development (including lung development) and receptor signaling and serine/threonine kinase pathways. The performance of a subset of antibodies was confirmed by ELISA using an expanded sample set including subjects with benign nodules, with sensitivities ranging from 5-10% at 98% specificity.

We reported a 5-AAb panel (TTC14, BRAF, ACTL6B, MORC2, CTAG1B) that had 30% sensitivity at 89% specificity to distinguish lung cancer from high-risk controls with smoking histories. A comparison of AAb responses between lung cancer and patients with CT positive pulmonary nodules revealed a related but different 5-AAb panel (TTC14, BRAF, KLF8, TLK1, KRT8) with a sensitivity of 30% at 88% specificity.

We also calculated the sensitivities and specificities of all 19 candidate antigens that went into the validation study restricting benign samples with above or equal to 30 pack-year smoking history. All five reported candidate antigens still presented sensitivities above 5% at 98% specificity showing that they still provide discrimination between lung adenocarcinoma and benign controls (not shown). Although these panels require further validation, they do provide information on the complementarities of these informative antigens. Further analysis revealed that AAbs do not associate with tumor size, stage, or smoking history.

It is still not clear which factors determine the development of these humoral immune responses. Assuming AAb responses were linked to tissue overexpression of the corresponding protein target, only a small fraction of patients with the overexpressed protein will develop AAb responses at detectable level. We also examined the mRNA level of these AAb targets in TCGA lung adenocarcinoma data set. mRNA expressions of 6 out of the 8 proteins in both panels were significantly increased in lung adenocarcinoma tissues. This finding not only suggested that the development of AAbs in lung adenocarcinoma might be a result of protein overexpression, but also orthogonally verified the association of these AAbs to lung adenocarcinoma.

Strengths of this study include the use of a large number of plasma samples from adenocarcinoma of NSCLC with primarily stage I disease matched with smoker controls as well as controls with CT positive benign lung disease. We also used highly reproducible protein arrays for unbiased high-throughput screening of AAb candidates, which revealed informative pathways related to developmental processes and kinase signaling.

To evaluate these AAbs' performance, we used more clinically relevant ELISA assays in large sample sets, and performed independent blind validation. Our results were also consistent with TCGA mRNA expression data. A limitation of this study is that we did not randomize our patients at the beginning of the study into discovery and validation sets. This resulted more late stage samples in the validation sample set. Future validation studies with early stage samples are necessary to confirm the performance of our markers. Although this is one of the largest autoantibody studies to date in lung cancer, and the only one using patients and benign controls detected by CT screening, our sample size is still small to draw definitive conclusions in the multivariate analysis. Our study focused on a histologically homogeneous patient population with adenocarcinoma. However, we acknowledge future studies need to evaluate AAb responses and disease heterogeneity in subtypes of adenocarcinoma stratified by both their histological subtypes as well as molecular subtypes. In addition, the study relied on a proteome scale screen of wild type proteins for lung cancer-linked antibodies. As cDNA clones encoding proteins with known cancer-linked mutations become available, it may be useful to include those as well.

In summary, we have performed an immune-proteomic screening of AAb responses using protein arrays, and identified two panels of AAb that can potentially differentiate lung adenocarcinoma from smoker controls as well as CT positive benign lung disease. BRAF, as a putative oncogene, was also found to elicit humoral immune responses in lung cancer patients. For this study, we focused on markers with high specificity so that high risk subjects with a positive CT screen and a positive serum test should get more invasive test such as needle biopsy for their timely cancer diagnosis.

The following claims are not meant to be limited to the particular embodiments and examples herein.

What is claimed is:

1. A method comprising contacting a patient sample with a panel of antigens to allow for auto-antibody-antigen complexes to form, wherein said panel of antigens comprises Tetratricopeptide Repeat Domain 14 (TTC14) and at least two antigens selected from the group consisting of: Serine/threonine-protein kinase B-raf (BRAF), ATPase MORC2 (MORC2), and Cancer/testis antigen 1 (NYESO1, CTAG1B); and
detecting whether each of TTC14 and the at least two antigens forms an auto-antibody-antigen complex in the patient sample.

2. The method of claim 1, wherein said panel of antigens comprises TTC14, BRAF, ACTL6B, MORC2, and CTAG1B.

3. The method of claim 1, wherein said panel of antigens comprises TTC14, BRAF, KLF8, TLK1, and KRT8.

4. The method of claim 3, wherein said panel of antigens and a control provides discrimination between lung adenocarcinoma and benign cells.

5. The method of claim 1, further comprising obtaining the patient sample from a needle biopsy from the patient.

6. A method of confirming or questioning a lung cancer diagnosis, comprising the step of:
using a patient sample, comparing an AAb response to a panel of antigens with those of lung cancer patients and patients with CT positive pulmonary nodules, wherein said panel of antigens comprises TTC14, BRAF, KLF8, TLK1, and KRT8.

7. A kit for detecting lung cancer, comprising the panel of antigens of claim 2 and instructions for contacting a patient sample with said panel and detecting formation of antigen-auto-antibody complexes in comparison to a control.

8. A kit for detecting lung cancer, comprising the panel of antigens of claim 3 and instructions for contacting a patient sample with said panel and detecting formation of antigen-auto-antibody complexes in comparison to said control.

9. The method of claim 1, wherein the panel of antigens comprises one or more additional antigens selected from the group consisting of: Vacuolar protein sorting-associated protein 72 homolog (VPS72), CTTNBP2 N-terminal-like protein (CTTNBP2NL), Testis-specific Y-encoded-like protein (TSPYL2), Activin receptor type-2B (ACVR2B), Krueppel-like factor 8 (KLF8), G patch domain and ankyrin repeat-containing protein 1 (BAT4), Golgin subfamily A member 2 (C12ORF50), IQ domain-containing protein E (IQCE), Centrosome and spindle pole-associated protein 1 (CSPP1), Keratin, type II cytoskeletal 8 (KRT8), Protein FAM76A (FAM76A), Merlin (NF2), Serine/threonine-protein kinase tousled-like 1 (TLK1), and Cellular tumor antigen p53 (P53), and wherein the method further comprises detecting whether at least one of the additional antigens forms an auto-antibody-antigen complex in the patient sample.

10. The method of claim 1, further comprising contacting a control sample with the panel of antigens;
detecting whether at least one of the antigens of the panel forms an auto-antibody-antigen complex in the control sample;
and diagnosing the patient with lung cancer when the patient sample has increased auto-antibody-antigen complex levels compared to the control.

11. The method of claim 10, further comprising treating the patient with an immunotherapy to treat lung cancer.

12. The method of claim 10, wherein prior to the treating step the patient has a positive computed tomography (CT) screen for signs of lung cancer.

13. The method of claim 10, wherein the control sample is obtained from a cancer-free smoker.

14. A method comprising contacting a patient sample with a panel of antigens to allow for auto-antibody-antigen complexes to form, wherein said panel of antigens comprises five antigens comprising: Tetratricopeptide Repeat Domain 14 (TTC14), Actin-like protein 6B (ACTL6B), Serine/threonine-protein kinase B-raf (BRAF), ATPase MORC2 (MORC2), and Cancer/testis antigen 1 (NYESO1, CTAG1B); and
detecting whether at least four of the five antigens forms an auto-antibody-antigen complex in the patient sample.

15. The method of claim 14, wherein the panel of antigens consists of Tetratricopeptide Repeat Domain 14 (TTC14), Actin-like protein 6B (ACTL6B), Serine/threonine-protein kinase B-raf (BRAF), ATPase MORC2 (MORC2), and Cancer/testis antigen 1 (NYESO1, CTAG1B).

16. The method of claim 14, further comprising contacting a control sample with the panel of antigens;
detecting whether at least one of the five antigens forms an auto-antibody-antigen complex in the control sample;
and diagnosing the patient with lung cancer when the patient sample has increased auto-antibody-antigen complex levels compared to the control sample.

17. The method of claim 14, further comprising treating the patient with an immunotherapy to treat lung cancer.

18. The method of claim 14, wherein prior to the treating step the patient has a positive computed tomography (CT) screen for signs of lung cancer.

19. The method of claim 14, wherein the control sample is obtained from a cancer-free smoker.

20. The method of claim 14, wherein the method comprises detecting whether each of the five antigens forms an auto-antibody-antigen complex in the patient sample.
